(19) United States
(12) Patent Application Publication
(10) Pub. No.: US 2020/0297615 A1
(43) Pub. Date: Sep. 24, 2020

(54) ANTI-SETTLING, THICKENING POLYMER AND AQUEOUS CLEANSING FORMULATIONS CONTAINING SAME

(71) Applicants: Rohm and Haas Company, Collegeville, PA (US); Dow Global Technologies LLC, Midland, MI (US)

(72) Inventors: Kinjalbahen Joshi, Collegeville, PA (US); Fanwen Zeng, Audubon, PA (US); Wei Gao, Fort Washington, PA (US); Inna Shulman, Langhorne, PA (US); Curtis Schwartz, Ambler, PA (US); Adam Peter Jarvis, Liverpool (GB); Nicholas John Ainger, Wallasey (GB); Sally Elizabeth Wood, Warington (GB); Pierre Starck, Chester (GB); Neil Scott Shaw, Warrington (GB)

(21) Appl. No.: 16/756,062

(22) PCT Filed: Oct. 22, 2018
(86) PCT No.: PCT/US2018/056846

§ 371 Date: Apr. 14, 2020

Related U.S. Application Data
(60) Provisional application No. 62/581,162, filed on Nov. 3, 2017.

(51) Int. Cl. A61K 8/81; A61K 8/58; C08F 220/18; A61Q 5/02

(52) U.S. Cl. CPC A61K 8/8147; A61K 8/8152; A61K 8/585; A61K 2800/48; A61Q 5/02; A61K 2800/10; C08F 220/18

(57) ABSTRACT

An anti-settling, thickening polymer is provided for use in an acidic aqueous cleansing formulation having a pH of <5, wherein the anti-settling, thickening polymer, comprises: (a) structural units of C₁₋₄ alkyl acrylate; (b) structural units of methacrylic acid; (c) structural units of 2-acrylamido-2-methylpropane sulfonic acid (AMPS); (d) structural units of a specialized associated monomer having the following structure

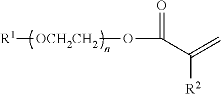

wherein R¹ is a linear saturated C₁₀₋₂₄ alkyl group; wherein R² is a hydrogen or a methyl; and wherein n is an average of 20 to 28; (e) structural units of acrylic acid; and (f) structural units of multi-ethylenically unsaturated crosslinking monomer or chain transfer agent; and wherein the sum of the weight percentages of structural units (a)-(f) is equal to 100 wt % of the anti-settling, thickening polymer. Also provided are acidic aqueous cleansing formulations containing same.

The present invention relates to an anti-settling, thickening polymer for use in an acidic aqueous cleansing formulation and to acidic aqueous cleansing formulations containing same. In particular, the present invention relates to an anti-settling, thickening polymer for use in acidic aqueous cleansing formulations having a pH of <5 and to acidic aqueous cleansing formulations containing same, wherein the anti-settling, thickening polymer, comprises: (a) structural units of C₁₋₄ alkyl acrylate; (b) structural units of methacrylic acid; (c) structural units of 2-acrylamido-2-methylpropane sulfonic acid (AMPS); (d) structural units of a specialized associated monomer having the following structure

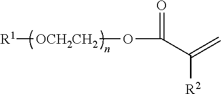

wherein R¹ is a linear saturated C₁₀₋₂₄ alkyl group; wherein R² is a hydrogen or a methyl group; and wherein n is an average of 20 to 28; with the proviso that the structural units of the specialized associated monomer (d) are derived from one of (i) a single specialized associated monomer; (ii) two specialized associated monomers, wherein R¹ is, respectively, a linear saturated C₁₂ and a linear saturated C₁₈ alkyl group; or (iii) two specialized associated monomers, wherein R¹ is, respectively, a linear saturated C₁₈ alkyl group and a linear saturated C₂₂ alkyl group; (e) structural units of acrylic acid; and (f) structural units of multi-ethylenically unsaturated crosslinking monomer or chain transfer agent; and wherein the sum of the weight percentages of structural units (a)-(f) is equal to 100%.

Aqueous cleansing compositions having insoluble materials suspended therein have desirability for a variety of conventional uses (e.g., personal care formulations, such as body washes, shampoos and conditioners). The insoluble materials typically impart, or contribute to, certain user benefits when incorporated into cleansing compositions, including: abrasion, visual product esthetics, various active effects (e.g., antidandruff properties) and encapsulation/release of separate phases during use. To be acceptable to consumers, such aqueous cleansing compositions desirably exhibit both an appealing look and feel. Such suspensions, however, in complex aqueous cleansing formulations for rinse-off applications in home and personal care applications present significant challenges, particularly under low pH acidic conditions.

Notwithstanding, the benefits associated with the incorporation of insoluble materials suspended in aqueous cleansing compositions, their incorporation creates a variety of complications. For example, insoluble materials typically have a density disparate from the continuous phase of the composition. This density mismatch can lead to compositional instability. In systems containing insoluble materials with a density less than that of the continuous phase, the insoluble materials tend to float to the top surface of the continuous phase (i.e., creaming). In systems containing insoluble materials with a density greater than that of the continuous phase, the insoluble materials tend to sink to the bottom of the continuous phase (i.e., settling). To further exacerbate the complications associated with the desirable incorporation of insoluble materials suspended in various aqueous cleansing compositions (for example, personal care formulations intended for application to skin), many of these compositions are desirably provided at a low pH (e.g., <5). As a result, conventional anti-settling, thickening polymers fail to provide adequate stability for such low pH compositions.

An approach to the suspending of insoluble materials in an aqueous cleansing formulation is disclosed in U.S. Pat. No. 5,154,847 to LaPetina, et al. LaPentina, et al. disclose an antidandruff shampoo comprising an anionic surfactant in an amount of about 5% to about 20% by weight; a particulate antidandruff agent in an amount of about 0.2% to about 5% by weight; a water insoluble suspending agent, solid at room temperature, selected from the group consisting of a suspending alkanolamide, a wax ester, and mixtures thereof, in an amount of about 1% to about 3% by weight; a crosslinked, neutralized polyacrylic acid resin in an amount of about 0.3% to about 1% by weight; and a liquid carrier.

Another approach to the suspending of insoluble materials in an aqueous cleansing formulation is disclosed in U.S. Pat. No. 8,642,056 to Souzy, et al. Souzy, et al. disclose a method for thickening a formulation, comprising contacting a cosmetic formulation with a direct aqueous emulsion of a polymer, followed by regulation of the pH to a value between 5 and 7, thereby forming a thickened formulation, wherein the emulsion is free from surfactants and organic solvents other than water and the polymer consists, expressed as a % by weight of each of the monomers therein, of: a) 20% to 60% by weight of methacrylic acid and/or acrylic acid, where the % by weight of acrylic acid, if present, compared to the total weight of acrylic acid and methacrylic acid is at least 50%, b) 40% to 80% by weight of at least one monomer chosen from among ethyl acrylate, butyl acrylate, and methyl methacrylate, c) 0.5% to 25% by weight of a monomer comprising a hydrophobic group, d) 0.05% to 22% by weight of 2-acrylamido-2-methylpropane sulfonic acid, and e) 0 to 1% by weight of at least one cross-linked monomer, wherein the monomer comprising a hydrophobic group has the general formula

R—(OE)ₘ-(OP)ₙ—R′,

m and n are integers of less than or equal to 150, at least one of which is non-zero, OE and OP are respectively ethylene oxide and propylene oxide, R is a polymerizable group selected from the groups consisting of methacrylate and methacrylurethane groups, R′ is a hydrophobic group having at least 6 and at most 36 carbon atoms.

Another approach to the suspending of insoluble materials in an aqueous cleansing formulation is disclosed in U.S. Pat. No. 6,106,816 to Hitchen. Hitchen discloses an aqueous conditioning shampoo composition comprising, in addition to water: (a) from 2 to 40% by weight of surfactant selected from the group consisting of anionic, nonionic and amphoteric surfactants, and mixtures thereof; (b) from 0.01 to 10% by weight of insoluble, non-volatile silicone which conditions hair; (c) from 0.01 to 3% by weight of titanium dioxide coated mica particles dispersed in the shampoo matrix; and (d) from 0.2 to 3% by weight of a crosslinked acrylic acid polymer for suspending the dispersed titanium dioxide coated mica particles and preventing them from settling in the composition as well as the insoluble, non-volatile silicone conditioning agent from creaming to the top of the composition on standing.

Notwithstanding, there remains a continuing need for an anti-settling, thickening polymers for use in acidic aqueous cleansing formulations having a pH of <5 and that incorporate insoluble materials; wherein the anti-settling, thickening polymer maintains the incorporated insoluble materials in suspension and wherein the acidic aqueous cleansing formulation also exhibits desirable rheology and aesthetic characteristics.

The present invention provides an anti-settling, thickening polymer for use in an acidic aqueous cleansing formulation having a pH of <5, wherein the anti-settling, thickening polymer, comprises: (a) 40 to 74.5 wt % of structural units of C₁₋₄ alkyl acrylate; (b) 20 to 50 wt % of structural units of methacrylic acid; (c) 0.2 to <5 wt % of structural units of 2-acrylamido-2-methylpropane sulfonic acid (AMPS); (d) 5 to 25 wt % of structural units of a specialized associated monomer having the following structure

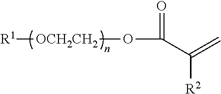

wherein R¹ is a linear saturated C₁₀₋₂₄ alkyl group; wherein R² is a hydrogen or a methyl group (preferably, wherein R² is a methyl group); and wherein n is an average of 20 to 28; with the proviso that the structural units of the specialized associated monomer (d) are derived from one of (i) a single specialized associated monomer (preferably, a single specialized associated monomer wherein R¹ is selected from the group consisting of a linear saturated C₁₂ alkyl group, a linear saturated Cis alkyl group and a linear saturated C₂₂ alkyl group; more preferably, a single specialized associated monomer wherein R¹ is selected from the group consisting of a linear saturated C₁₂ alkyl group and a linear saturated Cis alkyl group); (ii) two specialized associated monomers, wherein R¹ is, respectively, a linear saturated C₁₂ and a linear saturated C₁₈ alkyl group; or (iii) two specialized associated monomers, wherein R¹ is, respectively, a linear saturated C₁₈ alkyl group and a linear saturated C₂₂ alkyl group; (e) 0 to 1 wt % of structural units of acrylic acid; and (f) 0 to 2 wt % of structural units of multi-ethylenically unsaturated crosslinking monomer or chain transfer agent; and wherein the sum of the weight percentages of structural units (a)-(f) is equal to 100 wt % of the anti-settling, thickening polymer.

The present invention provides an acidic aqueous cleansing formulation, comprising: an anti-settling, thickening polymer of the present invention; a surfactant selected from the group consisting of anionic surfactants, nonionic surfactants, zwitterionic surfactants and mixtures thereof; and at least one insoluble additive; wherein the acidic aqueous cleansing formulation has a pH of <5.

The present invention provides an acidic aqueous cleansing formulation, comprising: an anti-settling, thickening polymer of the present invention; a surfactant selected from the group consisting of anionic, nonionic, zwitterionic surfactants and mixtures thereof; at least one insoluble additive; wherein the at least one insoluble additive is selected from the group consisting of a silicone, a gaseous bubble, an anti-dandruff agent, almond meal, apricot seed powder, barley flour, corn cob meal, corn cob powder, corn flour, corn meal, corn starch, oat bran, oat flour, oatmeal, peach pit powder, pecan shell powder, jojoba seed powder, pumice, rice bran, rye flour, soy flour, walnut shell powder, wheat bran, wheat flour, wheat starch, lufah, clay, Fuller's earth, alumina, aluminum oxide, aluminum silicate, palygorskite, bismuth oxychloride, boron nitride, calcium carbonate, calcium phosphate, calcium pyrophosphate, calcium sulfate, cellulose chalk, chitin, diatomaceous earth, dicalcium phosphate, dicalcium phosphate dihydrate, hydrated silica, hydroxyapatite, kaolin, iron oxide, magnesium trisilicate, tin oxide, mica, titanium dioxide, titanium dioxide coated mica, tricalcium phosphate zirconium silicate, microcrystalline cellulose, montmorillonite, polybutylene, polyethylene, polyisobutylene, polymethylstyrene, polypropylene, polystyrene, polyurethane, nylon, polytetrafluoroethylene, polyhalogenated olefins, hydrogenation products of jojoba oil, interesterification products of jojoba oil, sericite, silica, silk, sodium bicarbonate, sodium silicoaluminate, synthetic hectorite, talc, wax, resin and mixtures thereof; and wherein the acidic aqueous cleansing formulation has a pH of <5.

### DETAILED DESCRIPTION

We have now found that anti-settling, thickening polymers, as described herein, facilitate the formulation of acidic aqueous cleansing formulations with a pH of <5 and having a uniform dispersion in aqueous solution of an insoluble additive throughout for an indefinite period of time without negatively degrading the viscosity, foaming or cleaning properties of the cleansing formulation. Still further, the anti-settling, thickening polymers, as described herein, facilitate the formulation of acidic aqueous cleansing formulations with a pH of <5 and having a uniform dispersion of both an insoluble additive and an insoluble, non-volatile cosmetically acceptable silicone throughout for an indefinite period of time; wherein the anti-settling, thickening polymer of the present invention simultaneously inhibits the insoluble, non-volatile cosmetically acceptable silicone from rising to the top surface of the formulation and the insoluble additive from either rising to the top surface of the formulation or precipitating out of the formulation.

Unless otherwise indicated, ratios, percentages, parts, and the like are by weight.

As used herein, unless otherwise indicated, the phrase “molecular weight” or Mw refers to the weight average molecular weight as measured using asymmetric flow field flow fractionation (AF4) with inline Multi-Angle Light Scattering (MALS) and differential Refractive Index (RI) detections. The AF4 instrument used consisted of an Eclipse™ DualTec™ separation system (from Wyatt Technology Corp.) that was coupled in series to an 18 angle multi-angle light scattering (MALS) detector (DAWN HELOS II; from Wyatt Technology Corp.) and a differential refractometer (RI) (Optilab rEX; from Wyatt Technology Corp.). Flows through the AF4 instrument were provided using an Agilent Technologies 1200 series isocratic pump equipped with a micro-vacuum degasser. All injections were performed with an auto sampler (Agilent Technologies 1200 series). Data from the AF4 instrument were collected and processed using Astra software version 7.0.1.23 (from Wyatt Technology Corp.). Samples were prepared at a concentration of 1 mg/mL in 20 mM ammonium acetate solution at pH 10 (filtered with a 1.2 am pore nylon membrane). Samples (25 μL) were injected into the standard separation channel system (25 cm long and a width dimension starting at 2.15 cm and reducing to 0.3 cm over the length) with a channel thickness of 350 m and equipped with a 10 kDA cut of regenerated cellulose ultrafiltration membrane (Wyatt Technology). The mobile phase used for the AF4 analysis was 20 mM ammonium acetate solution at pH 10. Separation was performed with an applied channel flow of 1 mL/min. The sample was introduced to the channel with a focus flow at 1.7 mL/min for 3 minutes. The elution flow as then started at 0.5 mL/min for 3 minutes and then followed by a linearly decreasing cross flow gradient (from 0.5 mL/min to 0.05 mL/min over 12 minutes), then a hold at 0.05 mL/min for another 5 minutes. The average molecular weight was calculated using Astra software version 7.0.1.23 after subtracting a blank injection with a refractive index increment (dn/dc) of 0.190 mL/g for all calculation with Berry model 2^{nd} order fit. Molecular weights are reported herein in units of Daltons.

The term “polymer” as used herein and in the appended claims refers to a compound prepared by polymerizing monomers, whether of the same or a different type. The generic term “polymer” includes the terms “homopolymer,” “copolymer,” and “terpolymer.”

Percentages of monomer units in a polymer are percentages of solids or neat monomer weight, i.e., excluding any water present in a polymer emulsion.

The term “cosmetically acceptable” as used herein and in the appended refers to ingredients typically used in personal care compositions, and is intended to underscore that materials that are toxic when present in the amounts typically found in personal care compositions are in hair care compositions are not contemplated as part of the present invention.

The term “structural units” as used herein and in the appended claims refers to the remnant of the indicated monomer; thus a structural unit of ethyl acrylate is illustrated:

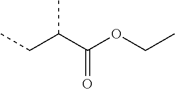

where the dotted lines represent the points of attachment to the polymer backbone.

The term “insoluble” as used herein and in the appended claims in reference to the insoluble additive means that the additive is essentially insoluble in the acidic aqueous cleansing formulation of the present invention. In particular, the insoluble additives have a solubility of less than 1 gram per 100 grams (preferably, less than 0.5 grams per 100 grams; more preferably, 0.1 grams to 100 grams) of the acidic aqueous cleansing formulation at 25° C.

The term “aesthetic characteristics” as used herein and in the appended claims in reference to an acidic aqueous cleansing formulation refers to visual and tactile sensory properties (e.g., smoothness, tack, lubricity, texture, color, clarity, turbidity, uniformity).

The term “aqueous” as used herein and in the appended claims in reference to an acidic aqueous cleansing formulation means that the formulation contains a sufficient amount of water to swell or dissolve the anti-settling, thickening polymer incorporated into the formulation.

Preferably, the anti-settling, thickening polymer of the present invention for use in an acidic aqueous cleansing formulation having a pH of <5 (preferably, 3.0 to 4.9; more preferable, 3.5 to 4.8; most preferably, 3.75 to 4.75), comprises: (a) 40 to 74.5 wt % (preferably, 45 to 69.5 wt %; more preferably, 50 to 65 wt %; most preferably, 55 to 61 wt %) of structural units of C₁₋₄ alkyl acrylate (preferably, C₂₋₄ alkyl acrylate; more preferably, C₂₋₃ alkyl acrylate; most preferably, ethyl acrylate); (b) 20 to 50 wt % (preferably, 25 to 45 wt %; more preferably, 25 to 40 wt %; most preferably, 30 to 35 wt %) of structural units of methacrylic acid; (c) 0.2 to <5 wt % (preferably, 0.5 to 3 wt %; more preferably, 0.75 to 2.0 wt %; most preferably, 0.75 to 1.5 wt %) of structural units of 2-acrylamido-2-methylpropane sulfonic acid (AMPS); (d) 5 to 25 wt % (preferably, 7.5 to 22.5 wt %; more preferably, 10 to 20 wt %; most preferably, 12.5 to 18 wt %) of structural units of a specialized associated monomer having the following structure

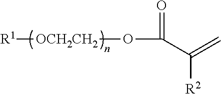

wherein R¹ is a linear saturated C₁₀₋₂₄ alkyl group; wherein R² is a hydrogen or a methyl group (preferably, wherein R² is a methyl group); and wherein n is an average of 20 to 28; with the proviso that the structural units of the specialized associated monomer (d) are derived from one of (i) a single specialized associated monomer (preferably, a single specialized associated monomer wherein R¹ is selected from the group consisting of a linear saturated C₁₂ alkyl group, a linear saturated C₁₈ alkyl group and a linear saturated C₂₂ alkyl group; more preferably, a single specialized associated monomer wherein R¹ is selected from the group consisting of a linear saturated C₁₂ alkyl group and a linear saturated Cis alkyl group); (ii) two specialized associated monomers, wherein R¹ is, respectively, a linear saturated C₁₂ and a linear saturated C₁₈ alkyl group; or (iii) two specialized associated monomers, wherein R¹ is, respectively, a linear saturated C₁₈ alkyl group and a linear saturated C₂₂ alkyl group; (e) 0 to 1 wt % (preferably, 0 to 0.1 wt %; more preferably, 0 to 0.01 wt %; most preferably, 0) of structural units of acrylic acid; and (f) 0 to 2 wt % (preferably, 0 to 0.1 wt %; more preferably, 0 to 0.001 wt %; most preferably, 0 wt %) of structural units of multi-ethylenically unsaturated crosslinking monomer and chain transfer agent; wherein the sum of the weight percentages of structural units (a)-(f) is equal to 100 wt % of the anti-settling, thickening polymer.

Preferably, the anti-settling, thickening polymer of the present invention, comprises: (a) 50 to 65 wt % of structural units of ethyl acrylate; (b) 25 to 40 wt % of structural units of methacrylic acid; (c) 0.75 to 2.0 wt % of structural units of 2-acrylamido-2-methylpropane sulfonic acid (AMPS); (d) 10 to 20 wt % of structural units of the specialized associated monomer; (e) 0 to 0.1 wt % of structural units of acrylic acid; and (f) 0 to 0.1 wt % (preferably, 0 to 0.001 wt %; more preferably, 0 wt %) of structural units of multi-ethylenically unsaturated crosslinking monomer and chain transfer agent; wherein the sum of the weight percentages of structural units (a)-(f) is equal to 100 wt % anti-settling, thickening polymer.

Preferably, the anti-settling, thickening polymer of the present invention has a weight average molecular weight of 5,000,000 to 400,000,000 Daltons. More preferably, the anti-settling, thickening polymer of the present invention has a weight average molecular weight of 25,000,000 to 300,000,000 Daltons. Most preferably, the anti-settling, thickening polymer of the present invention has a weight average molecular weight of 175,000,000 to 275,000,000 Daltons.

Preferably, the structural units of C₁₋₄ alkyl acrylate in the anti-settling, thickening polymer of the present invention are structural units of C₂₋₄ alkyl acrylate. More preferably, the structural units of C₁₋₄ alkyl acrylate in the anti-settling, thickening polymer of the present invention are structural units of C₂₋₃ alkyl acrylate. Most preferably, the structural units of C₁₋₄ alkyl acrylate in the anti-settling, thickening polymer of the present invention are structural units of ethyl acrylate.

Preferably, the anti-settling, thickening polymer of the present invention comprises 40 to 74.5 wt % of structural units of C₁₋₄ alkyl acrylate (preferably, C₂₋₄ alkyl acrylate; more preferably, C₂₋₃ alkyl acrylate; most preferably, ethyl acrylate). More preferably, the anti-settling, thickening polymer of the present invention comprises 45 to 69.5 wt % of structural units of C₁₋₄ alkyl acrylate (preferably, C₂₋₄ alkyl acrylate; more preferably, C₂₋₃ alkyl acrylate; most preferably, ethyl acrylate). Still more preferably, the anti-settling, thickening polymer of the present invention comprises 50 to 65 wt % of structural units of C₁₋₄ alkyl acrylate (preferably, C₂₋₄ alkyl acrylate; more preferably, C₂₋₃ alkyl acrylate; most preferably, ethyl acrylate). Most preferably, the anti-settling, thickening polymer of the present invention comprises 55 to 61 wt % of structural units of C₁₋₄ alkyl acrylate (preferably, C₂₋₄ alkyl acrylate; more preferably, C₂₋₃ alkyl acrylate; most preferably, ethyl acrylate).

Preferably, the anti-settling, thickening polymer of the present invention comprises 20 to 50 wt % of structural units of methacrylic acid. More preferably, the anti-settling, thickening polymer of the present invention comprises 25 to 45 wt % of structural units of methacrylic acid. Still more preferably, the anti-settling, thickening polymer of the present invention comprises 25 to 40 wt % of structural units of methacrylic acid. Most preferably, the anti-settling, thickening polymer of the present invention comprises 30 to 35 wt % of structural units of methacrylic acid.

Preferably, the anti-settling, thickening polymer of the present invention comprises 0.2 to <5 wt % of structural units of 2-acrylamido-2-methylpropane sulfonic acid (AMPS). More preferably, the anti-settling, thickening polymer of the present invention comprises 0.5 to 3 wt % of structural units of 2-acrylamido-2-methylpropane sulfonic acid (AMPS). Still more preferably, the anti-settling, thickening polymer of the present invention comprises 0.75 to 2.0 wt % of structural units of 2-acrylamido-2-methylpropane sulfonic acid (AMPS). Most preferably, the anti-settling, thickening polymer of the present invention comprises 0.75 to 1.5 wt % of structural units of 2-acrylamido-2-methylpropane sulfonic acid (AMPS).

Preferably, the anti-settling, thickening polymer of the present invention comprises 5 to 25 wt % of structural units of a specialized associated monomer having the following structure

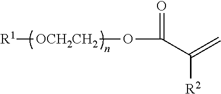

wherein R¹ is a linear saturated C₁₀₋₂₄ alkyl group; wherein R² is a hydrogen or a methyl group (preferably, wherein R² is a methyl group); and wherein n is an average of 20 to 28; with the proviso that the structural units of the specialized associated monomer (d) are derived from one of (i) a single specialized associated monomer (preferably, a single specialized associated monomer wherein R¹ is selected from the group consisting of a linear saturated C₁₂ alkyl group, a linear saturated C₁₈ alkyl group and a linear saturated C₂₂ alkyl group; more preferably, a single specialized associated monomer wherein R¹ is selected from the group consisting of a linear saturated C₁₂ alkyl group and a linear saturated Cis alkyl group); (ii) two specialized associated monomers, wherein R¹ is, respectively, a linear saturated C₁₂ and a linear saturated Cis alkyl group; or (iii) two specialized associated monomers, wherein R¹ is, respectively, a linear saturated C₁₈ alkyl group and a linear saturated C₂₂ alkyl group. More preferably, the anti-settling, thickening polymer of the present invention comprises 7.5 to 22.5 wt % of structural units of a specialized associated monomer having the following structure

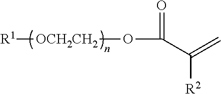

wherein R¹ is a linear saturated C₁₀₋₂₄ alkyl group; wherein R² is a hydrogen or a methyl group (preferably, wherein R² is a methyl group); and wherein n is an average of 20 to 28; with the proviso that the structural units of the specialized associated monomer (d) are derived from one of (i) a single specialized associated monomer (preferably, a single specialized associated monomer wherein R¹ is selected from the group consisting of a linear saturated C₁₂ alkyl group, a linear saturated C₁₈ alkyl group and a linear saturated C₂₂ alkyl group; more preferably, a single specialized associated monomer wherein R¹ is selected from the group consisting of a linear saturated C₁₂ alkyl group and a linear saturated Cis alkyl group); (ii) two specialized associated monomers, wherein R¹ is, respectively, a linear saturated C₁₂ and a linear saturated C₁₈ alkyl group; or (iii) two specialized associated monomers, wherein R¹ is, respectively, a linear saturated C₁₈ alkyl group and a linear saturated C₂₂ alkyl group. Still more preferably, the anti-settling, thickening polymer of the present invention comprises 10 to 20 wt % of structural units of a specialized associated monomer having the following structure

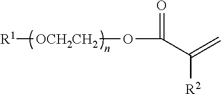

wherein R¹ is a linear saturated C₁₀₋₂₄ alkyl group; wherein R² is a hydrogen or a methyl group (preferably, wherein R² is a methyl group); and wherein n is an average of 20 to 28; with the proviso that the structural units of the specialized associated monomer (d) are derived from one of (i) a single specialized associated monomer (preferably, a single specialized associated monomer wherein R¹ is selected from the group consisting of a linear saturated C₁₂ alkyl group, a linear saturated C₁₈ alkyl group and a linear saturated C₂₂ alkyl group; more preferably, a single specialized associated monomer wherein R¹ is selected from the group consisting of a linear saturated C₁₂ alkyl group and a linear saturated Cis alkyl group); (ii) two specialized associated monomers, wherein R¹ is, respectively, a linear saturated C₁₂ and a linear saturated Cis alkyl group; or (iii) two specialized associated monomers, wherein R¹ is, respectively, a linear saturated C₁₈ alkyl group and a linear saturated C₂₂ alkyl group. Most preferably, the anti-settling, thickening polymer of the present invention comprises 12.5 to 18 wt % of structural units of a specialized associated monomer having the following structure

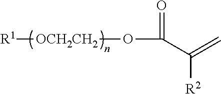

wherein R¹ is a linear saturated C₁₀₋₂₄ alkyl group; wherein R² is a hydrogen or a methyl group (preferably, wherein R² is a methyl group); and wherein n is an average of 20 to 28; with the proviso that the structural units of the specialized associated monomer (d) are derived from one of (i) a single specialized associated monomer (preferably, a single specialized associated monomer wherein R¹ is selected from the group consisting of a linear saturated C₁₂ alkyl group, a linear saturated C₁₈ alkyl group and a linear saturated C₂₂ alkyl group; more preferably, a single specialized associated monomer wherein R¹ is selected from the group consisting of a linear saturated C₁₂ alkyl group and a linear saturated Cis alkyl group); (ii) two specialized associated monomers, wherein R¹ is, respectively, a linear saturated C₁₂ and a linear saturated C₁₈ alkyl group; or (iii) two specialized associated monomers, wherein R¹ is, respectively, a linear saturated Cis alkyl group and a linear saturated C₂₂ alkyl group.

Preferably, the anti-settling, thickening polymer of the present invention includes 0 to 1 wt % of structural units of acrylic acid. More preferably, the anti-settling, thickening polymer of the present invention includes 0 to 0.1 wt % of structural units of acrylic acid. Still more preferably, anti-settling, thickening polymer of the present invention contains 0 to 0.01 wt % of structural units of acrylic acid. Yet still more preferably, the anti-settling, thickening polymer of the present invention includes less than the detectable limit of structural units of acrylic acid. Most preferably, the anti-settling, thickening polymer of the present invention contains 0 wt % structural units of acrylic acid.

Preferably, the anti-settling, thickening polymer of the present invention comprises 0 to 2 wt % of structural units of multi-ethylenically unsaturated crosslinking monomer and chain transfer agent. More preferably, the anti-settling, thickening polymer of the present invention comprises 0 to 0.1 wt % of structural units of multi-ethylenically unsaturated crosslinking monomer and chain transfer agent. Still more preferably, the anti-settling, thickening polymer of the present invention comprises 0 to 0.001 wt % of structural units of multi-ethylenically unsaturated crosslinking monomer and chain transfer agent. Yet still more preferably, the anti-settling, thickening polymer of the present invention includes less than the detectable limit of structural units of multi-ethylenically unsaturated crosslinking monomer and chain transfer agent. Most preferably, the anti-settling, thickening polymer of the present invention contains 0 wt % structural units of multi-ethylenically unsaturated crosslinking monomer and chain transfer agent.

Preferably, the anti-settling, thickening polymer of the present invention includes less than 0.001 wt % of structural units of multi-ethylenically unsaturated crosslinking monomer. More preferably, the anti-settling, thickening polymer of the present invention includes less than 0.0001 wt % of structural units of multi-ethylenically unsaturated crosslinking monomer. Still more preferably, anti-settling, thickening polymer of the present invention contains less than the detectable limit of structural units of multi-ethylenically unsaturated crosslinking monomer. Most preferably, the anti-settling, thickening polymer of the present invention includes 0 wt % of structural units of multi-ethylenically unsaturated crosslinking monomer.

Preferably, the anti-settling, thickening polymer of the present invention includes less than 0.1 wt % of structural units of chain transfer agent. More preferably, the anti-settling, thickening polymer of the present invention includes less than 0.001 wt % of structural units of chain transfer agent. Still more preferably, anti-settling, thickening polymer of the present invention contains less than the detectable limit of structural units of chain transfer agent. Most preferably, the anti-settling, thickening polymer of the present invention includes 0 wt % of structural units of chain transfer agent.

Preferably, the anti-settling, thickening polymer of the present invention includes less than 0.001 wt % of structural units of multi-ethylenically unsaturated crosslinking monomer and less than 0.1 wt % of structural units of chain transfer agent. More preferably, the anti-settling, thickening polymer of the present invention includes less than 0.0001 wt % of structural units of multi-ethylenically unsaturated crosslinking monomer and less than 0.01 wt % of structural units of chain transfer agent. Still more preferably, anti-settling, thickening polymer of the present invention contains less than the detectable limit of structural units of multi-ethylenically unsaturated crosslinking monomer and less than the detectable limit of structural units of chain transfer agent. Most preferably, the anti-settling, thickening polymer of the present invention includes 0 wt % of structural units of multi-ethylenically unsaturated crosslinking monomer and includes 0 wt % of structural units of chain transfer agents.

One of ordinary skill in the art will know to select appropriate multi-ethylenically unsaturated crosslinking monomers to provide any structural units of multi-ethylenically unsaturated crosslinking monomer in the anti-settling, thickening polymer of the present invention. Structural units of multi-ethylenically unsaturated crosslinking monomer may include for example those derived from polyunsaturated monomer components including, polyunsaturated aromatic monomers (e.g., divinyl benzene, divinyl naphthalene, trivinyl benzene); polyunsaturated alicyclic monomers (e.g., 1,2,4-trivinylcyclohexane); difunctional esters of phthalic acid (e.g., diallyl phthalate); polyunsaturated aliphatic monomers (e.g., isoprene, butadiene, 1,5-hexadiene, 1,5,9-decatriene, 1,9-decadiene, 1,5-heptadiene); polyalkenyl ethers (e.g., trially pentaerythritol, diallyl pentaerythritol, diallyl sucrose, octaally sucrose, trimethylolpropane dially ether); polyunsaturated esters of polyalcohols or polyacids (e.g., 1,6-hexanediol di(meth)acrylate, tetramethylene tri(meth)acrylate, allyl acrylate, diallyl itaconate, diallyl fumarate, diallyl maleat, trimethylolpropane tri(meth)acrylate, trimethylolpropane di(meth)acrylate, polyethylene glycol di(meth)acrylate); alkylene bisacrylamides (e.g., methylene bisacrylamide, propylene bisacrylamide); hydroxy and carboxy derivatives of methylene bis-acrylamide (e.g., N,N′-bismethylol methylene bisacrylamide); polyethyleneglycol di(meth)acrylates (e.g., ethyleneglycol di(meth)acrylate, diethyleneglycol di(meth)acrylate, triethyleneglycol di(meth)acrylate); polyunsaturated silanes (e.g., dimethyldivinylsilane, methyltrivinylsilane, allyldimethylvinylsilane, diallydimethylsilane, tetravinylsilane); polyunsaturated stannanes (e.g., tetraallyl tin, diallyldimethyl tin) and the like.

One of ordinary skill in the art will know to select appropriate chain transfer agents to provide any structural units of chain transfer agents in the anti-settling, thickening polymer of the present invention. Structural units of chain transfer agents may monomer include those derived from a variety of thio and disulfide containing compounds (e.g., C₁₋₁₈ alkyl mercaptans, mercaptocarboxylic acids, mercaptocarboxylic esters, thioesters, C₁₋₁₈ alkyl disulfides, aryldisulfides, polyfunctional thiols); phosphites and hypophosphites; haloalkyl compounds (e.g., carbon tetrachloride, bromotrichloromethane) and unsaturated chain transfer agents (e.g., alpha-methylstyrene).

Preferably, the acidic aqueous cleansing formulation of the present invention, comprises: (preferably, 0.1 to 4 wt %; more preferably, 0.5 to 3.5 wt %; still more preferably, 1 to 3 wt %; most preferably, 1.5 to 2.5 wt %) of an anti-settling, thickening polymer according to the present invention; (preferably, 2 to 40 wt %; more preferably, 5 to 30 wt %; still more preferably, 7.5 to 25 wt %; most preferably, 10 to 20 wt %) of a surfactant selected from the group consisting of anionic, nonionic, zwitterionic surfactants and mixtures thereof; and (preferably, 0.01 to 20 wt %; more preferably, 0.05 to 10 wt %; still more preferably, 0.075 to 7.5 wt %; most preferably, 0.1 to 2 wt %) of at least one insoluble additive; wherein the acidic aqueous cleansing formulation has a pH of <5 (preferably, 3.0 to 4.9; more preferable, 3.5 to 4.8; most preferably, 3.75 to 4.75).

Preferably, the acidic aqueous cleansing formulation of the present invention can have rheological properties ranging from pourable liquids to non-pourable gels.

Preferably, the acidic aqueous cleansing formulation of the present invention has a pH of <5 (preferably, 3.0 to 4.9; more preferable, 3.5 to 4.8; most preferably, 3.75 to 4.75). Preferably, the acidic aqueous cleansing formulations of the present invention can be personal care cleansing products, health care cleansing products, household care cleansing products and the like. More preferably, the acidic aqueous cleansing formulation of the present invention, is a personal care cleansing formulation; wherein the personal care cleansing formulation has a pH of <5 (preferably, 3.0 to 4.9; more preferable, 3.5 to 4.8; most preferably, 3.75 to 4.75). More preferably, the acidic aqueous cleansing formulation of the present invention, is a personal care cleansing formulation selected from the group consisting of body washes and shampoos; wherein the personal care cleansing formulation has a pH of <5 (preferably, 3.0 to 4.9; more preferable, 3.5 to 4.8; most preferably, 3.75 to 4.75). Most preferably, the acidic aqueous cleansing formulation of the present invention, is a shampoo; wherein the shampoo has a pH of <5 (preferably, 3.0 to 4.9; more preferable, 3.5 to 4.8; most preferably, 3.75 to 4.75).

Preferably, the acidic aqueous cleansing formulation of the present invention, includes 0.1 to 4 wt % of an anti-settling, thickening polymer of the present invention. More preferably, the acidic aqueous cleansing formulation of the present invention, includes 0.5 to 3.5 wt % of an anti-settling, thickening polymer of the present invention. Still more preferable, the acidic aqueous cleansing formulation of the present invention, includes 1 to 3.0 wt % of an anti-settling, thickening polymer of the present invention. Most preferably, the acidic aqueous cleansing formulation of the present invention, includes 1.5 to 2.5 wt % of an anti-settling, thickening polymer of the present invention.

Preferably, the acidic aqueous cleansing formulation of the present invention, includes 0.1 to 4 wt % (preferably, 0.5 to 3.5 wt %; more preferably, 1 to 3.0 wt %; most preferably, 1.5 to 2.5 wt %) of an anti-settling, thickening polymer of the present invention; wherein the anti-settling, thickening polymer, comprises: (a) 40 to 74.5 wt % (preferably, 45 to 69.5 wt %; more preferably, 50 to 65 wt %; most preferably, 55 to 61 wt %) of structural units of C₁₋₄ alkyl acrylate (preferably, C₂₋₄ alkyl acrylate; more preferably, C₂₋₃ alkyl acrylate; most preferably, ethyl acrylate); (b) 20 to 50 wt % (preferably, 25 to 45 wt %; more preferably, 25 to 40 wt %; most preferably, 30 to 35 wt %) of structural units of methacrylic acid; (c) 0.2 to <5 wt % (preferably, 0.5 to 3 wt %; more preferably, 0.75 to 2.0 wt %; most preferably, 0.75 to 1.5 wt %) of structural units of 2-acrylamido-2-methylpropane sulfonic acid (AMPS); (d) 5 to 25 wt % (preferably, 7.5 to 22.5 wt %; more preferably, 10 to 20 wt %; most preferably, 12.5 to 18 wt %) of structural units of a specialized associated monomer having the following structure

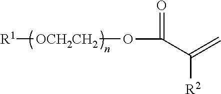

wherein R¹ is a linear saturated C₁₀₋₂₄ alkyl group; wherein R² is a hydrogen or a methyl group (preferably, wherein R² is a methyl group); and wherein n is an average of 20 to 28; with the proviso that the structural units of the specialized associated monomer (d) are derived from one of (i) a single specialized associated monomer (preferably, a single specialized associated monomer wherein R¹ is selected from the group consisting of a linear saturated C₁₂ alkyl group, a linear saturated C₁₈ alkyl group and a linear saturated C₂₂ alkyl group; more preferably, a single specialized associated monomer wherein R¹ is selected from the group consisting of a linear saturated C₁₂ alkyl group and a linear saturated Cis alkyl group); (ii) two specialized associated monomers, wherein R¹ is, respectively, a linear saturated C₁₂ and a linear saturated Cis alkyl group; or (iii) two specialized associated monomers, wherein R¹ is, respectively, a linear saturated C₁₈ alkyl group and a linear saturated C₂₂ alkyl group; (e) 0 to 1 wt % (preferably, 0 to 0.1 wt %; more preferably, 0 to 0.01 wt %; most preferably, 0) of structural units of acrylic acid; and (f) 0 to 2 wt % (preferably, 0 to 0.1 wt %; more preferably, 0 to 0.001 wt %; most preferably, 0) of structural units of multi-ethylenically unsaturated crosslinking monomer or chain transfer agent; wherein the sum of the weight percentages of structural units (a)-(f) is equal to 100 wt % of the anti-settling, thickening polymer (preferably, wherein the anti-settling, thickening polymer has a weight average molecular weight of 5,000,000 to 400,000,000 (more preferably, 25,000,000 to 300,000,000; most preferably, 175,000,000 to 275,000,000) Daltons).

Preferably, the surfactant used in the acidic aqueous cleansing formulation of the present invention is selected from the group of cosmetically acceptable surfactants. More preferably, the surfactant used in the acidic aqueous cleansing formulation of the present invention is selected from the group consisting of anionic surfactants, nonionic surfactants, zwitterionic surfactants and mixtures thereof.

Preferably, the acidic aqueous cleansing formulation of the present invention, includes 2 to 40 wt % of a surfactant selected from the group consisting of anionic, nonionic, zwitterionic surfactants and mixtures thereof. More preferably, the acidic aqueous cleansing formulation of the present invention, includes 5 to 30 wt % of a surfactant selected from the group consisting of anionic, nonionic, zwitterionic surfactants and mixtures thereof. Still more preferable, the acidic aqueous cleansing formulation of the present invention, includes 7.5 to 25 wt % of a surfactant selected from the group consisting of anionic, nonionic, zwitterionic surfactants and mixtures thereof. Most preferably, the acidic aqueous cleansing formulation of the present invention, includes 10 to 20 wt % of a surfactant selected from the group consisting of anionic, nonionic, zwitterionic surfactants and mixtures thereof.

Preferably, anionic surfactants used in the acidic aqueous cleansing formulation of the present invention are selected from the group of cosmetically acceptable anionic surfactants. Preferably, the anionic surfactants used in the acidic aqueous cleansing formulation of the present invention are selected from the group consisting of alkyl sulfates; alkyl ether sulfates; alkyl-substituted aryl sulfonates; alkyl succinates; alkyl sulfosuccinates; alkyl sarcosinates; α-olefin sulfonates; sodium, magnesium, ammonium, ethanolamine, diethanolamine and triethanolamine salts thereof; and mixtures thereof. More preferably, the anionic surfactants used in the acidic aqueous cleansing formulation of the present invention are selected from the group consisting of C₈₋₁₈ alkyl sulfates; C₈₋₁₈ alkyl (EO)ₙ(PO)ₘ sulfates, where n and m are independently 0 to 10 and where n+m is 1 to 10 (preferably, 2 to 3); C₈₋₁₈ alkyl-substituted aryl sulfonates; C₈₋₁₈ alkyl succinates; C₈₋₁₈ alkyl sulfosuccinates; C₈₋₁₈ alkyl sarcosinates; α-olefin sulfonates; sodium, magnesium, ammonium, ethanolamine, diethanolamine and triethanolamine salts thereof; and mixtures thereof. Still more preferably, the anionic surfactants used in the acidic aqueous cleansing formulation of the present invention are selected from the group consisting of sodium lauryl sulfate, sodium octadecyl succinate, ammonium lauryl sulphosuccinate, ammonium lauryl sulfate, ammonium lauryl ether sulfate, sodium dodecylbenzene sulfonate, triethanolamine dodecylbenzene sulfonate, sodium N-lauryl sarcosinate and mixtures thereof. Yet still more preferably, the anionic surfactants used in the acidic aqueous cleansing formulation of the present invention are selected from the group consisting of, sodium lauryl sulfate, sodium lauryl (EO)₂ sulfate, sodium lauryl (EO)₃ sulfate, ammonium lauryl sulfate, ammonium lauryl (EO) sulfate, ammonium lauryl (EO)₂ sulfate, ammonium (EO)₃ sulfate, triethanolamine dodecylbenzene sulfonate and mixtures thereof.

Preferably, nonionic surfactants used in the acidic aqueous cleansing formulation of the present invention are selected from the group of cosmetically acceptable nonionic surfactants. Preferably, the nonionic surfactants used in the acidic aqueous cleansing formulation of the present invention are selected from the group consisting of polyoxyalkylene surfactants, polyalkylene glycol esters, polyoxyethylene derivatives of fatty acid esters of polyhydric alcohols, fatty acid esters of polyalkoxylated polyhydric alcohols, polyalkoxylated natural fats and oils, polyalkylene oxide block copolymers, alkyl polyglucosides, sucrose esters and mixtures thereof. More preferably, the nonionic surfactants used are selected from polyoxyalkylene surfactants. Most preferably, the nonionic surfactants used are selected from polyoxyethylene surfactants. Preferred polyoxyethylene surfactants are selected from the group consisting of alcohol alkoxylates, alkylphenol alkoxylates and mixtures thereof. Preferred alcohol alkoxylates include, for example, alcohol ethoxylates and alcohol propoxylates. More preferred nonionic surfactants include nonionic surfactants selected from the group consisting of alcohol ethoxylate that conforms to the formula

R²—(OCH₂CH₂)_{w}OH

wherein R² is a C₁₀₋₃₀ alkyl group (preferably, a C₁₂₋₂₆ alkyl group; more preferably, a C₁₂₋₂₀ alkyl group; most preferably, a C₁₂₋₁₈ alkyl group); and w has an average value of 10 to 200 (preferably, 10 to 160; more preferably, 12 to 140; most preferably, 20 to 100). Still more preferred nonionic surfactants include nonionic surfactants selected from the group consisting of a polyethylene glycol ether of lauryl alcohol that conforms to the formula

CH₃(CH₂)₁₀CH₂(OCH₂CH₂)ₓOH

wherein x has an average value of 10 to 30 (preferably, 12 to 26; more preferably, 15 to 25; most preferably, 23); a polyethylene glycol ether of cetyl alcohol that conforms to the formula

CH₃(CH₂)₁₄CH₂(OCH₂CH₂)_{y}OH

wherein y has an average value of 10 to 30 (preferably, 12 to 26; more preferably, 15 to 25; most preferably, 20); a polyethylene glycol ether of stearyl alcohol that conforms to the formula

CH₃(CH₂)₁₆CH₂(OCH₂CH₂)_{z}OH

wherein z has an average value of 10 to 160 (preferably, 60 to 140; more preferably, 80 to 120; most preferably, 100); and mixtures thereof.

Preferably, zwitterionic surfactants used in the acidic aqueous cleansing formulation of the present invention are selected from the group of cosmetically acceptable zwitterionic surfactants. Preferably, the zwitterionic surfactants used in the acidic aqueous cleansing formulation of the present invention are selected from the group consisting of alkyl amine oxides, alkyl betaines, alkyl amido propyl betaines, alkyl alkanol amides, alkyl di-alkanol amides, alkyl sulfobetaines, alkyl glycinates, alkyl carboxy glycinates and mixtures thereof. More preferably, the zwitterionic surfactants used in the acidic aqueous cleansing formulation of the present invention are selected from the group consisting of C₈₋₁₈ alkyl amine oxides, C₈₋₁₈ alkyl betaines, C₈₋₁₈ alkyl amido propyl betaines, C₈₋₁₈ alkyl alkanol amides, C₈₋₁₈ alkyl di-alkanol amides, C₈₋₁₈ alkyl sulfobetaines, C₈₋₁₈ alkyl glycinates, C₈₋₁₈ alkyl carboxy glycinates and mixtures thereof. Preferred zwitterionic surfactants include lauryl amine oxide, cocamide monoethanolamine, cocamide diethanolamine, cocamidopropyl betaine, cocodimethyl sulfopropyl betaine and mixtures thereof.

Preferably, the acidic aqueous cleansing formulation of the present invention, includes any suitable amount of the at least one insoluble additive. More preferably, the acidic aqueous cleansing formulation of the present invention, includes 0.01 to 20 wt % of the at least one insoluble additive. More preferably, the acidic aqueous cleansing formulation of the present invention, includes 0.05 to 10 wt % of the at least one insoluble additive. Still more preferably, the acidic aqueous cleansing formulation of the present invention, includes 0.075 to 7.5 wt % of the at least one insoluble additive. Most preferably, the acidic aqueous cleansing formulation of the present invention, includes 0.1 to 2 wt % of the at least one insoluble additive.

Preferably, the at least one insoluble additive used in the acidic aqueous cleansing formulation of the present invention is selected from any of a variety of materials. The at least one insoluble additive can be hollow, porous, solid or some combination thereof. The at least one insoluble additive can be a two phase material (e.g., a first phase encapsulating a second phase). The at least one insoluble additive can be derived from at least one of an inorganic, an organic, a natural and a synthetic source.

Preferably, the at least one insoluble additive used in the acidic aqueous cleansing formulation of the present invention is selected from the group consisting of a silicone; a gaseous bubble; an anti-dandruff agent (e.g., zinc pyrithione, 2-hydroxypyridine 1-oxide); almond meal; apricot seed powder; barley flour; corn cob meal; corn cob powder; corn flour; corn meal; corn starch; oat bran; oat flour; oatmeal; peach pit powder; pecan shell powder; jojoba seed powder; pumice; rice bran; rye flour; soy flour; walnut shell powder; wheat bran; wheat flour; wheat starch; lufah; clay; Fuller's earth; alumina; aluminum oxide; aluminum silicate; palygorskite; bismuth oxychloride; boron nitride; calcium carbonate; calcium phosphate; calcium pyrophosphate; calcium sulfate; cellulose chalk; chitin; diatomaceous earth; dicalcium phosphate; dicalcium phosphate dihydrate; hydrated silica; hydroxyapatite; kaolin; iron oxide; magnesium trisilicate; tin oxide; mica; titanium dioxide; titanium dioxide coated mica; tricalcium phosphate zirconium silicate; microcrystalline cellulose; montmorillonite; polybutylene; polyethylene; polyisobutylene; polymethylstyrene; polypropylene; polystyrene; polyurethane; nylon; polytetrafluoroethylene; polyhalogenated olefins; hydrogenation products of jojoba oil; interesterification products of jojoba oil; sericite; silica; silk; sodium bicarbonate; sodium silicoaluminate; synthetic hectorite; talc; wax (e.g., paraffin, carnauba, ozocerite, candelilla); resin (e.g., urea-formaldehyde) and mixtures thereof. More preferably, the at least one insoluble additive used in the acidic aqueous cleansing formulation of the present invention is selected from the group consisting of a silicone; a gaseous bubble; an anti-dandruff agent (e.g., zinc pyrithione, 2-hydroxypyridine 1-oxide); hydrated silica; iron oxide; mica; titanium dioxide; titanium dioxide coated mica; hydrogenation products of jojoba oil; interesterification products of jojoba oil and mixtures thereof. Most preferably, the at least one insoluble additive used in the acidic aqueous cleansing formulation of the present invention is selected from the group consisting of a silicone, a gaseous bubble, mica, titanium dioxide, titanium dioxide coated mica, hydrogenated products of jojoba oil, interesterification products of jojoba oil and mixtures thereof.

Preferably, the at least one insoluble additive used in the acidic aqueous cleansing formulation of the present invention includes a silicone, wherein the silicone is an insoluble, non-volatile cosmetically acceptable silicone. Preferably, the insoluble, non-volatile cosmetically acceptable silicone is selected from the group consisting of amodimethicone, cyclomethicone, dimethicone, dimethiconol, hexadecyl methicone, hexamethyldisiloxane, methicone, phenyl dimethicone, stearoxy dimethicone polyalkyl siloxane, polyalkylaryl siloxane, silicone gums (i.e., polydiorganosiloxanes having a weight average molecular weight of 200,000 to 1,000,000 Daltons), polyaminofunctional silicones (e.g., Dow Corning® 929) and mixtures thereof. More preferably, the insoluble, non-volatile cosmetically acceptable silicone is selected from the group consisting of amodimethicone, cyclomethicone, dimethicone, dimethiconol, hexadecyl methicone, methicone and mixtures thereof. Still more preferably, the insoluble, non-volatile cosmetically acceptable silicone is selected from the group consisting of amodimethicone, dimethicone, dimethiconol and a mixture thereof. Most preferably, the cosmetically acceptable silicone includes dimethiconol.

Preferably, the insoluble, non-volatile cosmetically acceptable silicone is optionally incorporated into the formulation as a preformed emulsion (e.g., BY22-007, BY22-022 both available from Toray Silicone Co., Ltd.).

Preferably, the acidic aqueous cleansing formulation of the present invention, comprises: 0.01 to 20 wt % of an insoluble, non-volatile cosmetically acceptable silicone (preferably, wherein the insoluble, non-volatile cosmetically acceptable silicone conditions hair). Still more preferably, the acidic aqueous cleansing formulation of the present invention, further comprises 0.1 to 10 wt % of an insoluble, non-volatile cosmetically acceptable silicone (preferably, wherein the insoluble, non-volatile cosmetically acceptable silicone conditions hair). Yet still more preferably, the acidic aqueous cleansing formulation of the present invention, further comprises 0.1 to 5 wt % of an insoluble, non-volatile cosmetically acceptable silicone (preferably, wherein the insoluble, non-volatile cosmetically acceptable silicone conditions hair). Even more preferably, the acidic aqueous cleansing formulation of the present invention, further comprises 0.4 to 2.5 wt % of an insoluble, non-volatile cosmetically acceptable silicone (preferably, wherein the insoluble, non-volatile cosmetically acceptable silicone conditions hair). Most preferably, the acidic aqueous cleansing formulation of the present invention, further comprises 0.5 to 1.5 wt % of an insoluble, non-volatile cosmetically acceptable silicone (preferably, wherein the insoluble, non-volatile cosmetically acceptable silicone conditions hair).

Preferably, the at least one insoluble additive used in the acidic aqueous cleansing formulation of the present invention includes a first insoluble additive and a second insoluble additive; wherein the first insoluble additive is a silicone; wherein the silicone is an insoluble, non-volatile cosmetically acceptable silicone; and wherein the second insoluble additive is selected from the group consisting of a gaseous bubble, an anti-dandruff agent, almond meal, apricot seed powder, barley flour, corn cob meal, corn cob powder, corn flour, corn meal, corn starch, oat bran, oat flour, oatmeal, peach pit powder, pecan shell powder, jojoba seed powder, pumice, rice bran, rye flour, soy flour, walnut shell powder, wheat bran, wheat flour, wheat starch, lufah, clay, Fuller's earth, alumina, aluminum oxide, aluminum silicate, palygorskite, bismuth oxychloride, boron nitride, calcium carbonate, calcium phosphate, calcium pyrophosphate, calcium sulfate, cellulose chalk, chitin, diatomaceous earth, dicalcium phosphate, dicalcium phosphate dihydrate, hydrated silica, hydroxyapatite, kaolin, iron oxide, magnesium trisilicate, tin oxide, mica, titanium dioxide, titanium dioxide coated mica, tricalcium phosphate zirconium silicate, microcrystalline cellulose, montmorillonite, polybutylene, polyethylene, polyisobutylene, polymethylstyrene, polypropylene, polystyrene, polyurethane, nylon, polytetrafluoroethylene, polyhalogenated olefins, hydrogenation products of jojoba oil, interesterification products of jojoba oil, sericite, silica, silk, sodium bicarbonate, sodium silicoaluminate, synthetic hectorite, talc, wax, resin and mixtures thereof. More preferably, the second insoluble additive is selected from the group consisting of a gaseous bubble; an anti-dandruff agent (e.g., zinc pyrithione, 2-hydroxypyridine 1-oxide); hydrated silica; iron oxide; mica; titanium dioxide; titanium dioxide coated mica; hydrogenation products of jojoba oil; interesterification products of jojoba oil and mixtures thereof. Most preferably, the second insoluble additive is selected from the group consisting of a gaseous bubble, mica, titanium dioxide, titanium dioxide coated mica, hydrogenated products of jojoba oil, interesterification products of jojoba oil and mixtures thereof.

Preferably, the acidic aqueous cleansing formulation of the present invention, comprises water. More preferably, the acidic aqueous cleansing formulation of the present invention, comprises water, wherein the water generally comprises the balance of the acidic aqueous cleansing formulation, to bring the cationic hair care emulsion to 100 wt %, after the amounts of the other ingredients have been selected. Preferably, the water constitutes 5 to 39.9 wt % (preferably, 10 to 39.9 wt %; more preferably, 10 to 25 wt %) of the acidic aqueous cleansing formulation of the present invention.

Preferably, the acidic aqueous cleansing formulation of the present invention, further comprises: an optional ingredient selected from the group consisting of at least one of an antistatic agent; an antibacterial agent (e.g., phenoxyethanol, benzoic acid, benzyl alcohol, sodium benzoate, DMDM hydantoin, 2-ethylhexyl glyceryl ether, isothiazolinone); a foam booster; a perfume; a dye; a coloring agent; a preservative; a thickener (e.g., polysaccharides, cellulosic polymers); a protein; a film former; a phosphate ester; a cationic polymer (e.g., polyquaternium-10, polyquaterniums-24, polyquaternium-27, polyquaternium-67, polyquaternium-72); a buffering agent; a pH adjusting agent (e.g., citric acid, lactic acid, hydrochloric acid, aminoethyl propanediol, triethanolamine, monoethanolamine, sodium hydroxide, potassium hydroxide, amino-2-methyl-1-propanol) and mixtures thereof.

Preferably, the acidic aqueous cleansing formulation of the present invention, further comprises 0 to 1 wt % of a thickener. More preferably, the acidic aqueous cleansing formulation of the present invention, further comprises 0.1 to 1 wt % of a thickener. Still more preferably, the acidic aqueous cleansing formulation of the present invention, further comprises 0.2 to 0.7 wt % of a thickener. Most preferably, the acidic aqueous cleansing formulation of the present invention, further comprises 0.3 to 0.5 wt % of a thickener.

Preferably, the acidic aqueous cleansing formulation of the present invention, further comprises a pH adjusting agent. More preferably, the acidic aqueous cleansing formulation of the present invention, further comprises a pH adjusting agent, wherein the acidic aqueous cleansing formulation has a pH of <5 (preferably, 3.0 to 4.9; more preferable, 3.5 to 4.8; most preferably, 3.75 to 4.75). Most preferably, the acidic aqueous cleansing formulation of the present invention, further comprises a pH adjusting agent, wherein the acidic aqueous cleansing formulation has a pH of <5 (preferably, 3.0 to 4.9; more preferable, 3.5 to 4.8; most preferably, 3.75 to 4.75); wherein the pH adjusting agent is selected from the group consisting of citric acid, lactic acid, hydrochloric acid, aminoethyl propanediol, triethanolamine, monoethanolamine, sodium hydroxide, potassium hydroxide, amino-2-methyl-1-propanol and mixtures thereof (more preferably, wherein the pH adjusting agent is selected from the group consisting of citric acid, lactic acid, hydrochloric acid and mixtures thereof; most preferably, wherein the pH adjusting agent is hydrochloric acid).

Some embodiments of the present invention will now be described in detail in the following Examples.

### Comparative Example PC and Example P1-P2: Polymer Synthesis

In each of Comparative Example PC and Examples P1-P2, a 3 liter, 4 necked round bottom flask equipped with a mechanical stirrer, thermocouple, condenser and nitrogen sparge was charged with 430 g of deionized water and 4.7 g of sodium lauryl sulfate. The flask was then purged with nitrogen and its contents were warmed to 90° C. Then a first initiator solution containing 0.33 g of ammonium persulfate dissolved in 10 g of deionized water was added to the flask. Then a monomer solution was gradually charged to the flask over a period of 107 minutes, wherein the monomer solution contained 633 g deionized water, 18 g of sodium lauryl sulfate and the amounts (as noted in TABLE 1) of each of ethyl acrylate (EA), methacrylic acid (MAA), 2-acrylamido-2-methylpropane sulfonic acid (AMPS) and at least one lipophilically modified monomer (LIPO) having the following structure

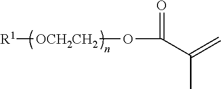

wherein R¹ is a linear saturated C₁₀₋₂₄ alkyl group; and wherein n is an average of 20 to 28. Starting simultaneously with the monomer solution charge, a second initiator solution containing 0.33 g of ammonium sulfate in 49 g of deionized water was gradually charged to the flask over a period of 112 minutes. Following the monomer charge and the second initiator solution charge, the transfer lines were rinsed with deionized water followed by a free radical catalyst and activator chase solution. The resulting latex products were recovered. The weight average molecular weight, Mw, of the recovered polymers measured by asymmetric flow field flow fractionation (AF4) are reported in TABLE 1.

TABLE 1

| | Monomer (g) | | | | | |
|---|---|---|---|---|---|---|
| Ex. | EA | MAA | LIPO¹ | LIPO² | AMPS | Mw (Daltons) |
| PC | 322 | 171 | 49 | — | 0 | 60,000,000 |
| P1 | 322 | 171 | 49 | — | 2.7 | 60,000,000 |
| P2 | 322 | 171 | 49 | — | 5.4 | 200,000,000 |
| P3 | 285 | 171 | 70 | 16 | 5.4 | — |
| P4 | 285 | 171 | 70 | 16 | 5.4 | — |
| P5 | 285 | 171 | 43 | 43 | 5.4 | — |

¹wherein R¹ is a linear saturated C₁₂ alkyl group
²wherein R¹ is a linear saturated C₁₈ alkyl group

### Comparative Example B1: Base Aqueous Cleansing Formulation

In Comparative Example B1, a base formulation was provided containing 48 g of a 25% solution of sodium laureth sulfate (Steol CS-230 SLES), 5.33 g of a 30% solution of cocamidopropyl betaine (Amphosol™ CA 30%); 20 g of a 1.5% solution of polyquaternium-10 (UCare™ JR-30M); 0.08 g methylchloroisothiazolinone/methylisothiazolinone (Kathon™ CG), wherein the base formulation pH was then adjusted to 4.5 through addition of hydrochloric acid and then the base formulation viscosity was adjusted to 5,000 to 8,000 cP through addition of sodium chloride.

### Comparative Example FC1: Aqueous Cleansing Formulation

The aqueous cleansing formulation of Comparative Example FC1 was prepared by dispersing 0.2 g of Flora pearls (Jojoba Esters) into a base formulation prepared according to Comparative Example B1.

### Examples F1-F4: Aqueous Cleansing Formulation

The aqueous cleansing formulations of Examples F1-F4 were prepared by dispersing 0.2 g of Flora pearls (Jojoba Esters) and a polymer product as noted in TABLE 2 into the base formulation prepared according to Comparative Example B1, wherein the aqueous cleansing formulations contained 2 wt % of the added polymer product.

TABLE 2

| Example | Polymer Product |
|---|---|
| F1 | Example P2 |
| F2 | Example P3 |
| F3 | Example P4 |
| F4 | Example P5 |

### Comparative Example B2: Base Aqueous Cleansing Formulation

In Comparative Example B2, a base formulation was provided by adding dimethiconol, TEA-dodecylbenzenesulfonate (Dow Corning 1784 available from The Dow Chemical Company) to a base formulation prepared according to Comparative Example B1, wherein the aqueous cleansing formulation of Comparative Example B2 contained 1.5 wt % of the added dimethiconol, TEA-dodecylbenzenesulfonate.

### Comparative Example FC2: Aqueous Cleansing Formulation

The aqueous cleansing formulation of Comparative Example FC2 was prepared by dispersing 0.2 g of Flora pearls (Jojoba Esters) into a base formulation prepared according to Comparative Example B2.

### Example F5: Aqueous Cleansing Formulation

The aqueous cleansing formulation of Example F5 was prepared by dispersing 0.2 g of Flora pearls (Jojoba Esters) and a polymer product prepared according to Example P2 into the base formulation prepared according to Comparative Example B2, wherein the aqueous cleansing formulation of Example F5 contained 2 wt % of the polymer product of Example P2.

### Heat Age Stability Testing

An aliquot (1 mL) of each of the base aqueous cleansing formulations prepared according to Comparative Examples B1-B2 and each of the aqueous cleansing formulations prepared according to Comparative Examples FC1-FC2 and Examples F1-F5 was added to a separate capped 1 mL clear glass vial (8 mm×43 mm). The samples were placed in an aluminum sample holder and heat aged for 10 days at 45° C. The heat aged samples were then pH adjusted to 4.5.

The Flora pearls (Jojoba Esters) in the heat aged cleansing formulation samples prepared according to Comparative Example FC1 and FC2 were in both cases observed to have completely risen to the surface of the sample near the liquid-gas interface. In addition, a liquid phase separation was also apparent in the silicone containing sample, Comparative Example FC2.

The Flora pearls (Jojoba Esters) in the heat aged cleansing formulation samples prepared according to Examples F1-F5 were all observed to have remained stably dispersed throughout the sample. In addition, no liquid phase separation was apparent in the silicone containing sample, Example F5.

We claim:

1. An anti-settling, thickening polymer for use in an acidic aqueous cleansing formulation having a pH of <5, wherein the anti-settling, thickening polymer, comprises:
(a) 40 to 74.5 wt % of structural units of C₁₋₄ alkyl acrylate;
(b) 20 to 50 wt % of structural units of methacrylic acid;
(c) 0.2 to <5 wt % of structural units of 2-acrylamido-2-methylpropane sulfonic acid (AMPS);
(d) 5 to 25 wt % of structural units of a specialized associated monomer having the following structure 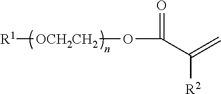
wherein R¹ is a linear saturated C₁₀₋₂₄ alkyl group; wherein R² is a hydrogen or a methyl; and wherein n is an average of 20 to 28, with the proviso that the structural units of the specialized associated monomer (d) are derived from (i) a single specialized associated monomer; (ii) two specialized associated monomers, wherein R¹ is, respectively, a linear saturated C₁₂ and a linear saturated Cis alkyl group; or (iii) two specialized associated monomers, wherein R¹ is, respectively, a linear saturated C₁₈ alkyl group and a linear saturated C₂₂ alkyl group;
(e) 0 to 1 wt % of structural units of acrylic acid; and
(f) 0 to 2 wt % of structural units of multi-ethylenically unsaturated crosslinking monomer or chain transfer agent; and
wherein the sum of the weight percentages of structural units (a)-(f) is equal to 100 wt % of the anti-settling, thickening polymer.

2. The anti-settling, thickening polymer of claim 1, wherein the anti-settling, thickening polymer includes less than 0.001 wt % of structural units of multi-ethylenically unsaturated crosslinking monomer; wherein the anti-settling, thickening polymer includes less than 0.1 wt % of structural units of chain transfer agent; and wherein the anti-settling, thickening polymer includes less than 0.1 wt % of structural units of acrylic acid.

3. The anti-settling, thickening polymer of claim 2, wherein the anti-settling, thickening polymer has a weight average molecular weight of 25,000,000 to 300,000,000 Daltons.

4. The anti-settling, thickening polymer of claim 1, wherein the anti-settling, thickening polymer includes:
(a) 50 to 65 wt % of structural units of C₁₋₄ alkyl acrylate, wherein the C₁₋₄ alkyl acrylate is ethyl acrylate;
(b) 25 to 40 wt % of structural units of methacrylic acid;
(c) 0.75 to 2.0 wt % of structural units of 2-acrylamido-2-methylpropane sulfonic acid (AMPS);
(d) 10 to 20 wt % of structural units of the specialized associated monomer;
(e) 0 to 0.1 wt % of structural units of acrylic acid; and
(f) 0 to 0.001 wt % of structural units of multi-ethylenically unsaturated crosslinking monomer or chain transfer agent.

5. An acidic aqueous cleansing formulation, comprising:
an anti-settling, thickening polymer according to claim 1;
a surfactant selected from the group consisting of anionic, nonionic, zwitterionic surfactants and mixtures thereof; and
at least one insoluble additive;
wherein the acidic aqueous cleansing formulation has a pH of <5.

6. The acidic aqueous cleansing formulation of claim 5, wherein the acidic aqueous cleansing formulation has a pH of 3.75 to 4.75.

7. The acidic aqueous cleansing formulation of claim 5, wherein the at least one insoluble additive is selected from the group consisting of a silicone, a gaseous bubble, an anti-dandruff agent, almond meal, apricot seed powder, barley flour, corn cob meal, corn cob powder, corn flour, corn meal, corn starch, oat bran, oat flour, oatmeal, peach pit powder, pecan shell powder, jojoba seed powder, pumice, rice bran, rye flour, soy flour, walnut shell powder, wheat bran, wheat flour, wheat starch, lufah, clay, Fuller's earth, alumina, aluminum oxide, aluminum silicate, palygorskite, bismuth oxychloride, boron nitride, calcium carbonate, calcium phosphate, calcium pyrophosphate, calcium sulfate, cellulose chalk, chitin, diatomaceous earth, dicalcium phosphate, dicalcium phosphate dihydrate, hydrated silica, hydroxyapatite, kaolin, iron oxide, magnesium trisilicate, tin oxide, mica, titanium dioxide, titanium dioxide coated mica, tricalcium phosphate zirconium silicate, microcrystalline cellulose, montmorillonite, polybutylene, polyethylene, polyisobutylene, polymethylstyrene, polypropylene, polystyrene, polyurethane, nylon, polytetrafluoroethylene, polyhalogenated olefins, hydrogenation products of jojoba oil, interesterification products of jojoba oil, sericite, silica, silk, sodium bicarbonate, sodium silicoaluminate, synthetic hectorite, talc, wax, resin and mixtures thereof.

8. The acidic aqueous cleansing formulation of claim 5, wherein the at least one insoluble additive includes a silicone, wherein the silicone is an insoluble, non-volatile cosmetically acceptable silicone.

9. The acidic aqueous cleansing formulation of claim 5, where the at least one insoluble additive includes a first insoluble additive and a second insoluble additive; wherein the first insoluble additive is a silicone; wherein the silicone is an insoluble, non-volatile cosmetically acceptable silicone; wherein the second insoluble additive is selected from the group consisting of a gaseous bubble, an anti-dandruff agent, almond meal, apricot seed powder, barley flour, corn cob meal, corn cob powder, corn flour, corn meal, corn starch, oat bran, oat flour, oatmeal, peach pit powder, pecan shell powder, jojoba seed powder, pumice, rice bran, rye flour, soy flour, walnut shell powder, wheat bran, wheat flour, wheat starch, lufah, clay, Fuller's earth, alumina, aluminum oxide, aluminum silicate, palygorskite, bismuth oxychloride, boron nitride, calcium carbonate, calcium phosphate, calcium pyrophosphate, calcium sulfate, cellulose chalk, chitin, diatomaceous earth, dicalcium phosphate, dicalcium phosphate dihydrate, hydrated silica, hydroxyapatite, kaolin, iron oxide, magnesium trisilicate, tin oxide, mica, titanium dioxide, titanium dioxide coated mica, tricalcium phosphate zirconium silicate, microcrystalline cellulose, montmorillonite, polybutylene, polyethylene, polyisobutylene, polymethylstyrene, polypropylene, polystyrene, polyurethane, nylon, polytetrafluoroethylene, polyhalogenated olefins, hydrogenation products of jojoba oil, interesterification products of jojoba oil, sericite, silica, silk, sodium bicarbonate, sodium silicoaluminate, synthetic hectorite, talc, wax, resin and mixtures thereof.

10. The acidic aqueous cleansing formulation of claim 9, further comprising:
an optional ingredient selected from the group consisting of at least one of an antibacterial agent, a foam booster, a perfume, a dye, a coloring agent, a preservative, a thickener, a protein, a phosphate ester and a buffering agent.
